(19) United States
(12) Patent Application Publication
(10) Pub. No.: US 2010/0234362 A1
(43) Pub. Date: Sep. 16, 2010

(54) USE OF DIAZOXIDE FOR SUPPRESSING THE PLASMA INSULIN LEVEL IN A MAMMAL

(76) Inventor: Johannes Mathijs Maria De Boer, Velp (NL)

Correspondence Address: FOLEY AND LARDNER LLP;SUITE 500, 3000 K STREET NW, WASHINGTON, DC, 20007, US

(21) Appl. No.: 12/734,002

(22) PCT Filed: Oct. 2, 2008
(86) PCT No.: PCT/NL2008/050629

§ 371 Date: May 7, 2010

Related U.S. Application Data
(60) Provisional application No. 60/977,423, filed on Oct. 4, 2007.

(30) Foreign Application Priority Data
Oct. 4, 2007 (EP) 07117924.6

(51) Int. Cl. A61K 31/5415; A61P 3/04; A61P 3/00

(52) U.S. Cl. 514/223.2

(57) ABSTRACT

The present invention relates to the use of a potassium channel activator in the manufacture of a medicament for suppressing the fasting plasma insulin level and/or postabsorptive insulin level in a mammal in need thereof, wherein the fasting and/or postabsorptive plasma insulin level is reduced to about 5 mU/l or less. The present invention also relates to the use of a potassium channel activator in the manufacture of a medicament for suppressing the fasting plasma insulin level and/or postabsorptive insulin level in a mammal in need thereof for treating or preventing obesity, obesity related disorders and conditions and other disorders and conditions related to weight gain in a mammal in need thereof, said method comprising orally administering to said mammal in need thereof a daily dosage of about 5 mg to about 1200 mg, calculated on a Diazoxide active weight basis.

### FIELD OF THE INVENTION

The present invention relates to a method for suppressing the fasting plasma and/or postabsorptive insulin levels in a mammal, in particular in a male mammal. The present invention also relates to a method for preventing or treating enhanced fasting and/or postabsorptive plasma insulin levels in a mammal, in particular in a male mammal.

### BACKGROUND OF THE INVENTION

Obesity, which can be defined as a body weight more than 20% above the ideal body weight or even better by a Body Mass Index (BMI; expressed as the ratio of the mammal's weight and the square of its length) of 30 kg/m² or higher (cf. World Health Organization. Technical report series 894: “Obesity: preventing and managing the global epidemic.”, Geneva, World Health Organization, 2000), is a rapidly increasing global problem that urgently needs to be controlled. Obesity causes or exacerbates many health problems, both independently and in association with other disorders. The medical problems associated with obesity, which can be serious and life-threatening, include hypertension, type 2 diabetes mellitus, elevated plasma insulin concentrations; insulin resistance, dyslipidemias, hyperlipidemia, endometrial, breast-, prostate- and colon-cancer, osteoarthritis, respiratory complications, cholelithiasis, gallstones, arteriosclerosis, heart disorder, abnormal heart rhythms, and heart arrythmias. Reference is for example made to US 2006/276549 and WO 2006/124506 to Abbot Laboratories.

Nowadays, three medical compounds are used for the treatment of obesity, i.e. Rimonabant (Acomplia®), Sibutramine (Meridia®) and Orlistat (Xenical®). Rimonabant is originally disclosed in U.S. Pat. No. 5,624,941 to Sanofi. Rimonabant is manufactured by Sanofi-Aventis and is a CB1 cannabinoid receptor antagonist. Sibutramine is originally disclosed in U.S. Pat. No. 4,929,629 to Boots. Sibutramine is manufactured by Abbott Laboratories and is a neurotransmitter reuptake inhibitor. The use of Sibutramine for treating obesity is disclosed in U.S. Pat. No. 6,538,034 to Sepracor Inc. Orlistat is originally disclosed in U.S. Pat. No. 4,598,089 to Roche. Orlistat is manufactured by Roche and is a pancreatic lipase inhibitor. The use of Orlistat in treating obesity is also disclosed in U.S. Pat. No. 4,598,089 to Roche. Rimonanbant induces a (placebo-subtracted) net weight loss of about 6 kg (Despres et al., “Effects of Rimonanbant on metabolic risk factors in overweight patients with dyslipidemia”, N. Eng. J. Med, 353, 2121-2134, 2005), whereas Sibutramine and Orlistat produce a net weight loss of about 5 and 3 kg, respectively (Despres et al., “Effects of Rimonibant on metabolic risk factors in overweight patients with dyslipidemia”, N. Eng. J. Med. 353, 2121-2134, 2005; Li et al., “Pharmacologic treatment of obesity”, Ann Intern. Med. 142, 532-546, 2005). Although weight reductions of this magnitude do produce some favourable metabolic effects, the improvements are modest and are insufficient in treating more obese mammals.

Combinations of the pharmaceutically active agents Rimonabant, Sibutramine or Orlistat with other active components are also known in the prior art. For example, U.S. Pat. No. 7,037,944 and U.S. Pat. No. 7,148,258, both to Sanofi-Aventis, teach the use of the combination of Sibutramine and a CB1 receptor antagonist for the treatment of obesity. US 2006/276549 to Abbott Laboratories discloses the combination of sibutramine and rimonabant to treat obesity and obesity related disorders. Another example is US 2007/142369 to Schering Corp., which discloses the combination of a Histamine H3 antagonist and an appetite suppressant selected from the group consisting of a CB1 antagonist (e.g. rimonabant), sibutramine, phentermine and topiramate for the treatment of obesity and obesity related disorders. US 2005/124660 to Solvay discloses the use of a combination of a pancreatic lipase inhibitor, e.g. Orlistat, and a CB1 receptor antagonist for the treatment of obesity. US 2006/269510 to Roche discloses the combination of a lipase inhibitor, preferably Orlistat, and a bile acid sequestrant, for treating obesity. US 2006/135471 to Roche discloses the combination of a of a lipase inhibitor and a glucomannan for the treatment of obesity. US2007/060532 to Fournier Laboratories Ireland Ltd. discloses the use of Orlistat and Metformin (an anti-diabetic drug) to treat patients suffering from obesity. US 2007/078179, also to Fournier Laboratories Ireland Ltd., discloses the use of a fibrate and Orlistat to treat patients suffering from obesity. However, the combinations do not seem to have been investigated in great detail yet and the existing evidence does not suggest significantly greater weight reduction compared to single-drug treatment (cf. Padwal, R. S. and Majumdar, S. R., The Lancet 369, 71-77, 2007).

Octreotide of Novartis (Sandostatin®) is originally disclosed in U.S. Pat. No. 4,395,403. Octreotide is an octapeptide that mimics natural somatostatin. Lustig, R. H. et al., Int J Obes. 30, 331-341, 2006 (“A multicenter, randomized, double blind, placebo-controlled, dose-finding trial of a long-acting formulation of octreotide in promoting weight loss in obese adults with insulin hypersecretion”) discloses that patients receiving 40 or 60 mg of octreotide LAR experienced statistically significant weight loss of 0.73 and 0.79 kg/m²

Diaxozide (Proglycem®), a chemical compound of the group of 1,2,4-benzothiadiazine-1,1-dioxide derivatives, is a potassium channel activator and is used in the treatment of hypertension. The synthesis and its application as an anti-hypertensive pharmaceutical agent is disclosed in U.S. Pat. No. 2,968,573 and U.S. Pat. No. 3,345,365, both to Schering Corp. Diaxozide is also known as a pharmaceutical agent for the treatment of secretory diarrhea (cf. U.S. Pat. No. 5,234,922 to the University of Iowa).

U.S. Pat. No. 5,234,922 to Paulsen discloses administering oral Diazoxide to an individual before ingestion of a food source in an amount effective to normalize the blood glucose and insulin levels. Diazoxide is administered in an amount from about 0.4 to about 0.8 mg/kg body weight before each meal. U.S. Pat. No. 5,234,922 discloses that low doses of Diazoxide, i.e. about 0.4 to about 0.8 mg/kg body weight, taken before each meal are effective to normalize blood glucose and insulin levels in subjects with a disturbance in the regulation of glucose and insulin levels that is characterised by the occurrence of postprandial hyperglycemia and reactive hypoglycemia. Paulsen does not provide any evidence that Diazoxide in this dose range is effective in the treatment of obesity or in suppressing the (fasting or peak) plasma insulin level.

Alemdazeh et al., “Beneficial Effect of Diazoxide in Obese Hyperunsulinemic Adults”, J. Clin. Endocrin. Metab. 83, 1911-1915, 1998, discloses the potential use of Diazoxide as a pharmaceutical agent to induce weight loss in humans. In a randomised, placebo-controlled trial with 24 hyperinsulinemic patients (predominantly women), it was found that a relatively low dose of Diazoxide, i.e. maximal 200 mg/day, divided in three dosages) for eight weeks resulted in a significant higher weight loss than placebo (9.8 vs. 5.0 kg).

Schreuder et al., “Diazoxide-mediated insulin suppression in obese men: a dose-response study”, Diab. Obes. Metab. 7, 239-245, 2005, disclose the short-term Diazoxide-mediated insulin suppression in men in a one week dose-response study in order to determine the optimal dose in the treatment of obesity. Oral dosages at 150 and 225 mg per day (divided in three dosages) had no significant effect on plasma glucose and insulin levels, whereas an oral dosage of 300 mg per day (divided in three dosages), however, reduced plasma insulin levels by about 20% without affecting fasting or postprandial glucose levels. Consequently, it was found that insulin suppression is dose dependent, although the effect was found to be less in obese men than in non-obese men. Schreuder et al. suggested that effective insulin suppression in obese men would at least require a daily dose of 3.2 to 4.2 mg/kg body weight, i.e. for men in the weight range of 100-125 kg about 300 to 500 mg/day.

However, Due et al., “No effect of inhibition of insulin secretion by diazoxide on weight loss in hyperinsulinaemic obese subjects during an 8-week weight-loss diet”, Diab. Obes. Metab. 9, 566-574, 2007, could not confirm the results of Alemdazeh et al. in a study of similar design and duration. Due et al. concluded that hyperinsulinaemia does not contribute to the maintenance of the obese state and that insulin secretion inhibition is presumably not a target for treating or preventing obesity. Due et al. also concluded from the study of Schreuder et al. that a daily dose of 3.2 to 4.2 mg/kg body weight would lead to worsen the hyperglycaemic state and would produce more undesired side-effects and would therefore not to be recommended in the treatment of obesity.

Van Boekel et al., Abstractbook 67^{th} Annual Scientific Session American Diabetes Association June 2007, Chicago—Abstract 2742-PO, discloses an open, uncontrolled study to weight-loss by Diazoxide-induced insulin suppression in obese men (fourteen patients; four patients were eventually excluded). The dose of Diazoxide was started at 150 mg/day (divided in three equal dosages) and was raised every month to ultimately 900 mg/day (divided in three equal dosages), until side effects occurred. After six months, the mean dosage was about 585 mg/day (plasma level of about 49 mg/l). Higher dosages were generally not tolerated due to side effects, in particular hyperglycaemia and edema. A body weight reduction was observed from about 114 kg to about 103 kg. However, the absolute suppressive effect on plasma insulin level is not disclosed.

U.S. Pat. No. 6,197,765 to Vardi and Morad disclose the application of Diazoxide in the treatment of syndrome-X and resulting complications including hyperlipidemia, hypertension, central obesity, hyperinsulinemia and impaired glucose intolerance. It is disclosed that Diazoxide mediated suppression of pancreatic insulin secretion is an effective treatment for “the metabolic syndrome” and for the prevention and treatment of diabetic complications in adult-onset diabetes mellitus. Hence, the invention disclosed in U.S. Pat. No. 6,197,765 is a combination treatment consisting of Diazoxide administered in increasing doses until endogenous insulinopenia appears which is then combined with administration of exogenous insulin.

WO 2006/026469 to Essentialis Inc. disloses oral, controlled release pharmaceutical formulations preferably comprising Diazoxide which may be used in the treatment of various disorders including diabetes and obesity. Paragraph [00382] suggests a daily dose of 100, 200 and 300 mg/day (divided in two dosages). Results are not disclosed.

WO 2006/045799 to Solvay discloses pharmaceutical compositions comprising a CB1 antagonist and a potassium channel activator, e.g. Diazoxide. On page 29 it is disclosed that (4S)-3-(4-chlorophenyl)-N′-[(4-chlorophenyl)sulfonyl]-N-methyl-4-phenyl-4,5-dihydro-1-H-pyrazole-1-carboximidamide (indicated as Compound A) is more effective than Diazoxide at comparable dosages in inhibiting insulin secretion.

Consequently, there is still a need for a more efficient and efficacious method for treating obesity.

It was surprisingly found that potassium channel activator-mediated, in particular Diazoxide-mediated plasma insulin suppression involving relatively high daily oral dosages of Diazoxide is very effective in reducing body weight in mammals to a clinically relevant degree of weight reduction when the plasma insulin concentrations were lowered to particular levels. If such levels were not reached, the effect of Diazoxide-mediated plasma insulin suppression on body weight reduction is far less.

### SUMMARY OF THE INVENTION

The present invention relates to the use of a potassium channel activator in the manufacture of a medicament for suppressing the fasting and/or postabsorptive plasma insulin level in a mammal in need thereof, wherein the fasting and/or postabsorptive plasma insulin level is reduced to about 5 mU/l or less.

### BRIEF DESCRIPTION OF THE FIGURES

FIG. 1 shows the increase of the daily Diazoxide dose per month.

FIG. 2 shows the increase of the Diazoxide plasma level per month.

FIG. 3 shows the decrease of body weight per month.

FIG. 4 shows thee decrease of abdominal circumference per month.

FIG. 5 shows the relationship between the change in body fat mass and fasting insulin levels as observed over a period of six months.

### DEFINITIONS

The verb “to comprise” as is used in this description and in the claims and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there is one and only one of the elements. The indefinite article “a” or “an” thus usually means “at least one”.

All dosages are given relative to a Diazoxide active weight basis, that is that every dosage of a potassium channel activator, e.g. in mg/kg or mg/kg/day, should be interpreted as whether the potassium channel activator was actually Diazoxide; the molecular weight of Diazoxide (7-chloro-3-methyl-4H-1,2,4-benzothiadiazine 1,1-dioxide) is 230.7 g/mol.

In this document, the term “postabsorptive”: means the period between meals that starts after the ingested foods have been absorbed from the small intestine and there is no longer uptake of intestinal food components into the bloodstream.

### DETAILED DESCRIPTION OF THE INVENTION

### The Potassium Channel Activator

According to the present invention, the potassium channel activator is preferably a non-selective potassium channel activator, which is preferably selected from the group consisting of a 1,2,4-benzothiadiazine-1,1-dioxide derivatives. Such derivatives include not only the neutral organic compounds, but also their pharmaceutically acceptable addition salts, hydrates, solvates and polymorphs.

The 1,2,4-benzothiadiazine-1,1-dioxide derivatives can be represented by the general formula (I):

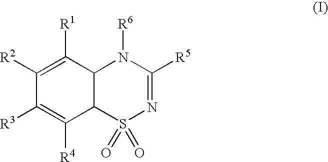

and tautomers thereof, wherein:
R¹, R², R³ and R⁴ are independently selected from the group consisting of hydrogen, halogen, C₁-C₆ alkyl and halogenated C₁-C₆ alkyl;
R⁵ and R⁶ are independently selected from the group consisting of hydrogen, C₁-C₆ alkyl, C₁-C₆ alkenyl and C₃-C₁₀ cycloalkyl.

Preferably, the 1,2,4-benzothiadiazine-1,1-dioxide derivatives are selected from the group consisting of the derivatives according to general formula (I), wherein R³ is a halogen, more preferably chlorine.

Its is also preferred that the 1,2,4-benzothiadiazine-1,1-dioxide derivatives are selected from the group consisting of the derivatives according to general formula (I), wherein R⁵ is a C₁-C₆ alkyl group, more preferably methyl.

Even more preferably, the 1,2,4-benzothiadiazine-1,1-dioxide derivatives are selected from the group consisting of:
7-chloro-3-methyl-4H-1,2,4-benzothiadiazine-1,1-dioxide (Diazoxide);
7-chloro-3-ethyl-4H-1,2,4-benzothiadiazine-1,1-dioxide;
6-chloro-3-methyl-4H-1,2,4-benzothiadiazine-1,1-dioxide;
3-n-propyl-6-trifluoromethyl-4H-1,2,4-benzothiadiazine-1,1-dioxide;
3-ethyl-6-trifluoromethyl-4H-1,2,4-benzothiadiazine-1,1-dioxide;
3-methyl-6-trifluoromethyl-4H-1,2,4-benzothiadiazine-1,1-dioxide;
7-bromo-3-isopropyl-4H-1,2,4-benzothiadiazine-1,1-dioxide;
6,7-dichloro-3-methyl-4H-1,2,4-benzothiadiazine-1,1-dioxide;
3-cyclopropyl-6,7-dichloro-4H-1,2,4-benzothiadiazine-1,1-dioxide;
7-chloro-3,6-dimethyl-4H-1,2,4-benzothiadiazine-1,1-dioxide;
6-chloro-3-n-butyl-4H-1,2,4-benzothiadiazine-1,1-dioxide;
6-chloro-3-n-pentyl-4H-1,2,4-benzothiadiazine-1,1-dioxide;
3,7-dimethyl-6-chloro-4H-1,2,4-benzothiadiazine-1,1-dioxide;
3-(3-cyclopentenyl)-6,7-dichloro-4H-4H-1,2,4-benzothiadiazine-1,1-dioxide;
6-chloro-3-(3-cyclopentenyl)-4H-1,2,4-benzothiadiazine-1,1-dioxide;
3-(3-cyclopentenyl)-6-trifluoromethyl-4H-1,2,4-benzothiadiazine-1,1-dioxide;
3-isopropylamino-4H-1,2,4-benzothiadiazine-1,1-dioxide;
3-isobutylamino-4H-1,2,4-benzothiadiazine-1,1-dioxide:
(2-ethylhexylamino)-4H-1,2,4-benzothiadiazine-1,1-dioxide;
cyclopropylamino-4H-1,2,4-benzothiadiazine-1,1-dioxide;
cyclohexylamino-4H-1,2,4-benzothiadiazine-1,1-dioxide;
7-chloro-3-(1,2,2-trimethylpropylamino)-4H-1,2,4-benzothiadiazine-1,1-dioxide;
7-chloro-3-(1,2-dimethylpropylamino)-4H-1,2,4-benzothiadiazine-1,1-dioxide:
7-chloro-3-(1-methylpropylamino)-4H-1,2,4-benzothiadiazine-1,1-dioxide;
7-chloro-3-isopropylamino-4H-1,2,4-benzothiadiazine-1,1-dioxide;
7-chloro-3-cyclopropylamino-4H-1,2,4-benzothiadiazine-1,1-dioxide;
7-chloro-3-cyclohexylamino-4H-1,2,4-benzothiadiazine-1,1-dioxide;
6-chloro-3-isopropylamino-4H-1,2,4-benzothiadiazine-1,1-dioxide;
6-chloro-3-cyclopropylamino-4H-1,2,4-benzothiadiazine-1,1-dioxide;
6,7-dichloro-3-isopropylamino-4H-1,2,4-benzothiadiazine-1,1-dioxide;
6,7-dichloro-3-cyclopropylamino-4H-1,2,4-benzothiadiazine-1,1-dioxide;
3-isobutylamino-7-methyl-4H-1,2,4-benzothiadiazine-1,1-dioxide;
3-cyclopentylamino-7-methyl-4H-1,2,4-benzothiadiazine-1,1-dioxide;
3-cyclohexylamino-6-trifluoromethyl-4H-1,2,4-benzothiadiazine-1,1-dioxide;
(N-cyclohexyl-N-methylamino)-4H-1,2,4-benzothiadiazine-1,1-dioxide;
3-cyclohexylamino-4-methyl-4H-1,2,4-benzothiadiazine-1,1-dioxide;
3-cyclohexylamino-2-methyl-2H-1,2,4-benzothiadiazine 1,1-dioxide;

Suitable 1,2,4-benzothiadiazine-1,1-dioxide derivatives are for example disclosed in U.S. Pat. No. 2,986,573, U.S. Pat. No. 3,345,365, U.S. Pat. No. 3,449,337, U.S. Pat. No. 3,462,428, U.S. Pat. No. 4,184,039 and U.S. Pat. No. 6,242,443, all incorporated by reference herein.

Most preferably, the potassium channel activator is Diazoxide.

### Therapeutic Applications

As already disclosed above, it was surprisingly found that the potassium channel activator-mediated plasma insulin suppression involving relatively high daily oral dosages of the potassium channel activator is very effective in reducing body weight in a mammal to a clinically relevant degree of weight reduction when the plasma insulin concentrations were lowered to particular levels. If such levels were not reached, the effect of the potassium channel activator-mediated plasma insulin suppression on body weight reduction is far less.

Accordingly, the potassium channel activator is administered to a mammal in need thereof for suppressing the fasting plasma insulin level in said mammal, wherein the fasting and/or postabsorptive plasma insulin level is reduced to about 5 mU/l or less.

According to a first preferred embodiment of the present invention, the potassium channel activator is administered to a mammal in need thereof to reduce or prevent weight gain. Consequently, the potassium channel activator may be administered systemic or prophylactic. Hence, the present invention also relates to a method for preventing or treating weight gain in a mammal in need thereof, said method comprising administering an amount of a potassium channel activator to said mammal. Preferably, the method according to this first preferred embodiment of the invention includes the reduction of the fasting and/or postabsorptive plasma insulin level to about 5 mU/l or less.

According to a second preferred embodiment of the present invention, the potassium channel activator is administered to a mammal in need thereof for the prevention or treatment of obesity, obesity related disorders and conditions and other disorders and conditions related to weight gain, wherein the potassium channel activator may be administered systemic or prophylactic. Obesity, obesity related disorders and conditions and said other disorders and conditions related to weight gain are usually the result of an increase of fatty tissue in the mammal to a certain point at which it is associated with certain health conditions or even mortality. Such obesity related disorders and conditions and said other disorders and conditions include cardiovascular disorders, diabetes, in particular diabetes mellitus type 2, sleep apnea and osteoarthritis. Accordingly, the present invention also relates to a method of preventing or treating obesity, obesity related disorders and conditions and other disorders and conditions related to weight gain in a mammal in need thereof, said method comprising administering an amount of a potassium channel activator to said mammal. Preferably, the method according to this second preferred embodiment of the invention includes the reduction of the fasting and/or postabsorptive plasma insulin level to about 5 mU/l or less.

### Methods of Administration

Obviously and if desired, the administration may be intermittently (or cyclic) or may be continuous. Generally, cyclic regimens of administration are characterized as being intermittent, as opposed to continuous treatment regimens, and have both treatment periods during which the potassium channel activator is administered and non-treatment periods to permit the systemic level of the potassium channel activator to return to baseline. For example, the administration may be continued for a period of six months followed by a period of two months where the potassium channel activator is not administered, where after the administration is started again for a period of six months. A continuous regimen of administration involves daily administration, twice-weekly administration, once-weekly administration, once every two weeks administration and the like.

According to the present invention, however, it is preferred that the potassium channel activator is administered continuously, preferably daily, in the form of an oral medicament to the mammal in need thereof.

It is also preferred according to the present invention that the potassium channel activator is administered to the mammal in need thereof for prolonged periods. Preferably, the potassium channel activator is administered for a period of one month to twenty four months, more preferably one month to twelve months.

According to a preferred embodiment of the present invention, the potassium channel activator is administered to the mammal in need thereof in such an mount that a serum level of the potassium channel activator is achieved of about 20 mg/l or more, calculated on a Diazoxide active weight basis, preferably 40 mg/l or more and even more preferably 45 mg/l or more. It is also preferred that the serum level of the potassium activator does not exceed 100 mg/l, calculated on a Diazoxide active weight basis, preferably 80 mg/l. Accordingly, the present invention also relates to a method for preventing or treating weight gain in a mammal in need thereof, said method comprising administering a potassium channel activator to said mammal in an amount sufficient to achieve a serum level of the potassium channel activator in said mammal of about 20 mg/l or more, calculated on a Diazoxide active weight basis. Likewise, the present invention also relates to a method of preventing or treating obesity, obesity related disorders and conditions and other disorders and conditions related to weight gain in a mammal in need thereof, said method comprising administering a potassium channel activator to said mammal in an amount sufficient to achieve a serum level of the potassium channel activator in said mammal of about 20 mg/l or more, calculated on a Diazoxide active weight basis. In both methods it is preferred that the serum level of the potassium activator does not exceed 100 mg/l, calculated on a Diazoxide active weight basis.

According to the present invention, the serum level of the potassium channel activator in said mammal of about 20 mg/l or more is maintained for at least one month, preferably for one month to twenty four months, even more preferably one month to twelve months.

Furthermore, the potassium channel activator may be administered to a mammal in need thereof in relatively low dosages according to cyclic or continuous administration regimens to reduce weight gain. The potassium channel activator may also be administered to a mammal in need thereof in relatively low dosages and according to cyclic or continuous administration regimens to prevent weight gain. Furthermore, the potassium channel activator may be administered to a mammal in need thereof in relatively low dosages and according to cyclic or continuous administration regimens to reduce weight gain or in the treatment of a disorder or condition associated with weight gain. Such disorders include obesity, diabetes and the like.

### Administration Schemes and Dosages

According to the invention, it is preferred that the potassium channel activator is administered to a mammal in need thereof over prolonged periods, preferably from one month to twenty four months, more preferably from one month to twelve months.

It is also preferred that the potassium channel activator or the medicament comprising the potassium channel activator is administered orally.

The dosages of the potassium channel activator need to be sufficient to achieve a serum level of the potassium channel activator in the mammal of about 20 mg/l or more as otherwise a reduction of the fasting and/or postabsorptive plasma insulin level to about 5 mU/l or less, is not achieved. Obviously, this is also dependent from the disorder or condition to be treated or prevented. Accordingly, the potassium channel activator is in general administered in a daily dosage of about 5 mg to about 1200 mg, calculated on a Diazoxide active weight basis. This implies that at a molar level the potassium channel activator is administered in a daily dosage of about 0.0217 mmol to about 5.202 mmol. However, it is preferred that the potassium channel activator is administered in a daily dosage of about 50 mg to about 900 mg, more preferably about 150 to about 900 mg, yet even more preferably about 300 mg to about 800 mg and most preferably about 400 mg to about 700 mg. These dosages are based on a BMI of a mammal of about 30 to about 35 kg/m².

According to a preferred embodiment of the invention, the mammal is administered a daily dosage of about 15 mg to about 300 mg, preferably about 75 mg to about 225 mg, more preferably about 100 mg to about 225 mg, even more preferably about 125 mg to about 225 mg, of the potassium channel activator, calculated on a Diazoxide active weight basis, for the first two to six weeks, wherein the dosage level is increased every two to six weeks with about 15 mg to about 300 mg, preferably about 75 mg to about 225 mg, more preferably about 100 mg to about 225 mg, even more preferably about 125 mg to about 225 mg, of the potassium channel activator. Preferably, the daily end-dosage does not exceed 1200 mg. If desired, these dosages may be divided over two or three dosages during the day. This dosage regimen enables the physician to control the serum level of the potassium channel activator and the fasting and/or postabsorptive insulin level during the prescription of the potassium channel activator, thereby enabling him to adjust the dosage level to an optimum for the mammal concerned. As it will be clear to the person skilled in the art, however, the dosage regimen is also dependent from the BMI of the mammal. Hence, mammals having a relatively lower BMI, in particular below 30 kg/m², but higher than 25 kg/m² (i.e. “overweight”; cf. World Health Organization. Technical report series 894: “Obesity: preventing and managing the global epidemic.”, Geneva, World Health Organization, 2000) may presumably need a lower dosage than mammals having a relatively higher weight, i.e. 30 kg/m² or higher. Consequently, in terms of BMI, the daily dosage is preferably about 5 mg to about 900 mg when the BMI of the mammal is 25-30 kg/m², more preferably about 50 mg to about 800 mg, even more preferably about 100 to about 800 mg, and most preferably 150 to about 700 mg. Obviously, such dosage regimen may also be intermittently (or cyclic) or may be continuous as is explained above. These dosage schemes may in particular be important for the prevention of disorders and conditions related to weight gain as is explained above.

### Pharmaceutical Compositions

Pharmaceutical compositions (or medicaments) comprising a potassium channel activator are known from the prior art. For example, Proglicem® is available as capsules containing 100 mg of Diazoxide. However, such pharmaceutical compositions comprise relatively low amounts of the potassium channel activator. For the medical uses indicated in this document, however, generally higher dosages are required. Accordingly, this invention also relates to pharmaceutical compositions comprising a potassium channel activator and a pharmaceutically acceptable carrier or excipient, wherein the pharmaceutical composition comprises about more than 100 mg, preferably 150 mg to about 1200 mg, calculated on a Diazoxide active weight basis. It is, however, preferred that the pharmaceutical composition comprises about 150 mg to about 900 mg, more preferably about 200 to about 900 mg, yet even more preferably about 300 mg to about 800 mg and most preferably about 400 mg to about 700 mg of the potassium channel activator. These pharmaceutical compositions are very suitable for daily administration. As it will be obvious to the person skilled in the art, such pharmaceutical compositions may also comprise only about one third of the active ingredient if the pharmaceutical composition is intended to be administered three times a day. Accordingly, the pharmaceutical composition or medicament may be administered once, two times or three times per day. Alternatively, if the pharmaceutical composition is intended to be administered in e.g. a once-weekly manner, the pharmaceutical composition may comprise the active ingredient in a relatively higher dosage.

### The Subject

According to the present invention, the mammal to be subjected to a treatment or a prevention of obesity, obesity related disorders and conditions and other disorders and conditions related to weight gain, is preferably human, more preferably male, and is most preferably a hyperinsulinemic obese man. It is also preferred that the fasting and/or postabsorptive plasma insulin level in said mammal is reduced to about 5 mU/l or less, wherein said mammal is subjected to such a (prophylactic or systemic) treatment.

### Example

### Subjects and Methods

Eighteen obese, healthy en were included in this study who were 30-50 years of age, had a BMI of above 30 to about 35 kg/m², a stable body weight for at least three months, a HbAlc<6.0% and a fasting C-peptide>1.0 nmol/l.

All subjects received were explained the physiological principle of this study. They also received dietary advice to reduce caloric intake to the calculated basal requirements for ideal body weight (based on the Harrison-Bennedict equation) and the instruction to increase walking exercise to at least 30 minutes a day. All subjects were allowed to do more physical exercise; the amount of daily physical exercise was not recorded. All subjects were instructed how to perform home glucose measurements (HGM). Blood samples were taken in the fasting state, (a) 2 hours after breakfast, just before lunch, (b) 2 hours after lunch, just before dinner, (c) 2 hours after dinner, at bedtime, and (d) at 3 am.

Baseline measurements included body weight, body height, abdominal circumference, body composition by total body dual energy X-ray absorptiometry (DEXA), supine and upright blood pressure, HGM for two days and the assessment of serum glucose levels and serum insulin levels in response to a standarised test meal. The meal test was performed after an overnight fast and started at 8.30 am (cf. T. Schreuder et al., “Diazoxide-mediated insulin suppression in obese men: a dose-response study”, Diab. Obes. Metab. 7, 239-245, 2005). Venous blood samples were taen through a venous line inserted into the forearm at −30, 0, 30, 45, 60, 90, 120, 180, 240 and 300 minutes. A 500 kCal test meal (about 2095 kJ) consisting of (parts by weight, calculated on the total weight of the test meal) about 50% carbohydrates, about 30% lipids and about 20% proteins was started at t₀ and was consumed within 15 minutes.

After completion of all baseline measurements, the subjects started Diazoxide 50 mg (three times a day) which was taken before each meal. Subsequently, the outpatient clinic was visited every month where the following data were recorded: body weight, abdominal circumference, supine and upright blood pressure, the results of a two-day, 8-point HGM performed in the week preceding the visit and adverse effects. Every visit, the daily Diazoxide dose was increased by 3×50 mg until a maximum of 300 mg (three times a day) was reached or until adverse side effects occurred. The Diazoxide dose was not increased if clinically relevant edema persisted for ore than a month, if upright systolic blood pressure was <110 mm Hg, if upright diastolic blood pressure was <70 mm Hg, or if fasting home glucose level was >7 nmol/l, or non-fasting home glucose level was >11 nmol/l.

After six months of treatment, all measurements were repeated including the meal test. Diazoxide was taken at t₋₃₀. Blood samples taken during the meal test at t₁₂₀ were also analysed for Diazoxide levels. Commercially available methods were used for measurement of plasma glucose (enzymatic colourimetric assay, p. 800 Roche Diagnostics, Mannheim, Germany), plasma insulin (electrochemiluminescence immuno assay, Elecsys 2010, Roche Diagnostics, Mannheim, Germany, manufacturer's reference range for fasting levels 0-20 mU/l) and plasma C-peptide concentration (competitive chemoluminescence immuno assay, DPC, Los Angeles, USA, manufarcturer's reference for fasting levels in non-obese subjects 0.15-1.00 nmol/l). Plasma Diazoxide levels were analysed by HPLC/UV (T. Schreuder et al., “Diazoxide-mediated insulin suppression in obese men: a dose-response study”, Diab. Obes. Metab. 7, 239-245, 2005). The results of total body DEXA were expressed as kg lean body mass, fat mass and bone mass. Normal values for serum glucose levels and serum insuli levels during the test meal were obtained by performing this test in ten healthy, age-matched, non-obese men (BMI about 20 to about 25 kg/m²).

All data are shown as mean values±standard error (SE). The paired t-test was used to analyse the response to Diazoxide treatment, employing the Bonferoni correction for multiple comparisons. A p-value of less than 0.05 was considered to be statistically significant.

### Results

Eighteen obese men with a mean BMI ranging from 31.3-34.7 kg/m² were included. Four men did not complete the study: 2 subjects showed low compliance and left the study prematurely for personal reasons, two men stopped because of side-effects (rash and edema, respectively).

Baseline characteristics and measurements of the 14 men completing the study according to protocol are summarized in Table 1. The estimated caloric intake prior to the study was 2242±125 kcal/day and comprised 48% carbohydrates, 32% fat, and 20% proteins. The recommended intake during the study was 1499±48 kcal/day. This represents a reduction of 31.3±3.4% compared to pre-study intake (P<0.001). The mean fasting insulin level at baseline was 18.0±2.3 mU/l and peak insulin concentration after the standardized meal was 124±16.8 mU/L. Both are about 3 times the normal value in age-matched, non-obese men. Mean HbAlc at baseline was 5.4±0.1%.

### DZX Dose and Side Effects

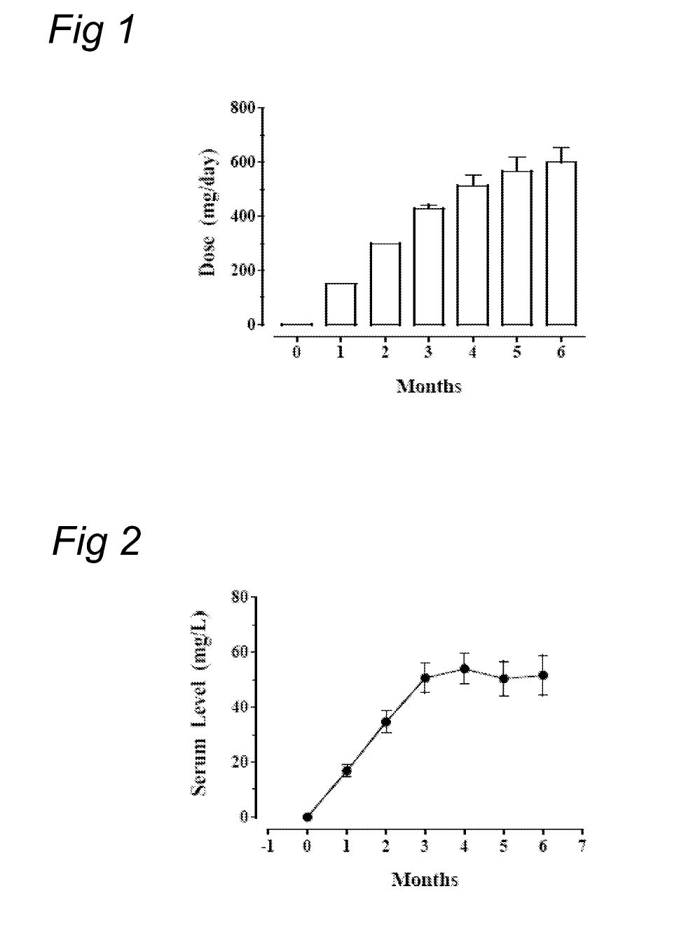

DZX was increased every month until a maximum of 900 mg/day was reached or until side effects occurred (FIG. 1). At six months the mean daily dose had increased to 600±54 mg (5.7±0.5 mg/kg), corresponding with a plasma DZX level of 50.0±5.8 mg/L (FIG. 2). Individually tolerated DZX doses ranged from 300 to 900 mg/day: 3 men tolerated only 300 mg/day, whereas only 2 men reached the 900 mg dose. Hyperglycaemia (n=4) and edema (n=8) were the main dose limiting events. Other adverse effects were not observed.

### Effects on Serum Glucose, Insulin, and Adiponectin Levels

As shown in Table 1, DZX reduced fasting insulin levels by 65% (P<0.001), post-meal insulin peak levels by 62% (P<0.001), and AUC_{Ins} by 63% (P<0.001). Fasting and peak glucoses increased by 0.8±0.3 mmol/L (P<0.01) and 1.4±0.7 mmol/L (P=0.06), respectively (FIG. 2b). The AUC_{Gluc} rose with 29% (P<0.02). HbAlc increased by 0.5% points (P<0.02). Glucosuria in fasting morning urine samples was not observed. Adiponectin levels increased by 100±17% (P<0.001). Serum adiponectin and fasting insulin levels were inversely related, before (R −0.67, P<0.01) and after (R −0.65, P=0.01) 6 months of treatment. However, the slopes of the lines defining these relationships were significantly different (P=0.025). Whole body insulin sensitivity increased by 209% (95% CI: 60-145%, P<0.001).

TABLE 1

| | Baseline | 6 Months | P-value |
|---|---|---|---|
| Age (yr) | 42.3 ± 1.9 | | |
| Height (cm) | 184 ± 2 | | |
| Weight (kg) | 115.1 ± 3.4 | 105.7 ± 3.5 | <0.001 |
| BMI (kg/m²) | 33.8 ± 0.5 | 31.0 ± 0.7 | <0.001 |
| Waist circumference (cm) | 116.9 ± 1.4 | 107.7 ± 2.2 | <0.001 |
| Systolic BP (mmHg) | 128.8 ± 3.2 | 128.8 ± 3.0 | NS |
| Diastolic BP (mmHg) | 85.8 ± 2.1 | 74.9 ± 2.1 | <0.001 |
| Fasting glucose (mmol/L) | 5.5 ± 0.2 | 6.3 ± 0.3 | <0.01 |
| Peak glucose (mmol/L) | 8.4 ± 0.3 | 9.8 ± 0.7 | NS |
| AUC glucose (mmol/L · min) | 1859 ± 56 | 2404 ± 195 | <0.02 |
| Fasting Insulin (mU/L) | 18.0 ± 2.3 | 5.6 ± 0.7 | <0.001 |
| Peak Insulin (mU/L) | 124 ± 17 | 35 ± 4 | <0.001 |
| AUC insulin (mU · 10⁻³/L · | 16.6 ± 2.1 | 5.4 ± 0.4 | <0.001 |
| min) | | | |
| HbA1c (%) | 5.4 ± 0.1 | 5.9 ± 0.2 | <0.02 |
| HDL-Cholesterol (mmol/L) | 1.12 ± 0.07 | 1.25 ± 0.09 | <0.05 |
| LDL-Cholesterol (mmol/L) | 3.19 ± 0.32 | 2.97 ± 0.23 | NS |
| Triglycerides (mmol/L) | 2.17 ± 0.26 | 1.08 ± 0.13 | <0.001 |
| Urate (mmol/L) | 0.37 ± 0.01 | 0.32 ± 0.02 | <0.02 |
| Adiponectin (mg/L) | 6.4 ± 0.5 | 12.5 ± 1.2 | <0.001 |
| Whole Body Insulin | 3.0 ± 0.3 | 8.8 ± 1.2 | <0.001 |
| Sensitivity | | | |
| Fat Mass (kg) | 40.1 ± 1.8 | 30.7 ± 2.1 | <0.001 |
| Soft Tissue LBM (kg) | 70.2 ± 2.0 | 70.6 ± 2.4 | NS |
| Bone Mass (kg) | 3.35 ± 0.14 | 3.47 ± 0.11 | NS |

BMI, body mass index;
BP, blood pressure;
AUC, area under the curve;
LBM, lean body mass.

### Effects on Body Composition

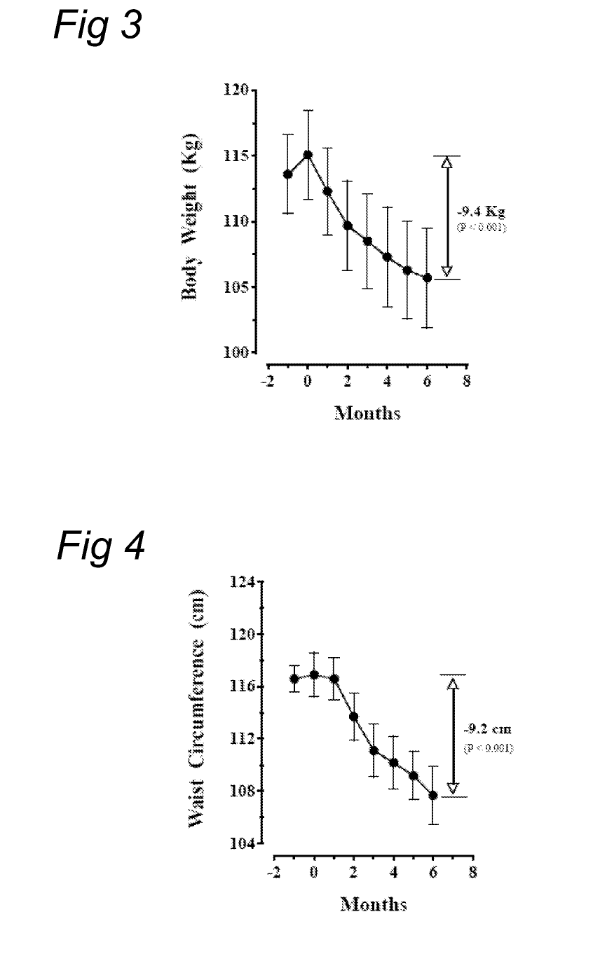

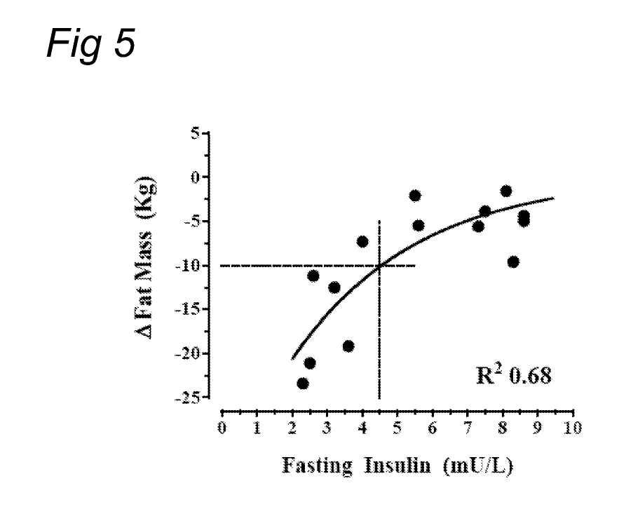

Body weight gradually decreased from 115.1±3.4 to 105.7±3.8 kg (−9.4 kg, −8.3%, P<0.001) (FIG. 3). Weight loss less than 5% was observed in 5/14 men (mean weight loss: 3.1±0.8%), between 5 en 10% in 4/14 men (mean weight loss: 6.7±0.5%), and more then 10% in 5/14 men (mean weight loss: 15.7±0.7%). Waist circumference decreased from 116.9±1.4 to 107.7±2.2 cm (−9.2 cm, −7.9%, P<0.001). Total body fat mass decreased by 9.5±1.9 kg (−23.3%, P<0.001), without a concomitant change in soft tissue lean body mass or bone mass (FIG. 4). The change in body weight was inversely correlated with fasting insulin levels at 6 months (R −0.59, P=0.023) and AUC_{Ins} (R −0.50, P 0.067). There was no correlation with post-meal peak insulin levels nor the change in fasting or peak insulin or AUC_{Ins} at 6 months. The change in fat mass as measured by DEXA was also related to the fasting insulin levels at 6 months (R −0.76, P=0.002) and AUC_{Ins} at 6 months (R −0.74, P=0.003), but not to peak insulin levels at 6 months or the change in fasting or peak insulin levels. As shown in FIG. 5, substantial loss of body fat, i.e. a decrease of 10 kg or more, only occurred if plasma insulin levels were below 4.5 mU/L, which is equivalent to about 30 pmol/L in this assay (conversion of mU/L to pmol/L: multiply by 6.945). The mean DZX dose in this subgroup was 630±56 mg.

### Effects on Blood Pressure, Lipids and Urate

Systolic blood pressure did not change. Diastolic blood pressure decreased by 10.9±2.3 mm Hg (95% CI: 6.5-15.4 mm Hg, P<0.001). Serum triglycerides decreased from 2.17±0.26 to 1.08±0.13 mmol/L (P<0.001). HDL cholesterol increased from 1.12±0.07 mmol/L to 1.25±0.09 mmol/L (P<0.05), whereas LDL cholesterol did not change significantly. Serum urate decreased from 0.37±0.01 to 0.32±0.02 mmol/L (P<0.02).

1.-16. (canceled)

17. A method of suppressing a fasting plasma insulin level, a post-absorptive insulin level, or both in a mammal in need of such suppression, said method comprising administering to said mammal a therapeutically effective amount of a potassium channel activator.

18. The method according to claim 17, wherein the fasting and/or post-absorptive plasma insulin is reduced to about 5 mU/l or less.

19. The method according to claim 17, wherein the potassium channel activator comprises a non-selective potassium channel activator.

20. The method according to claim 18, wherein the potassium channel activator comprises a 1,2,4-benzothiadiazine-1,1-dioxide derivative.

21. The method according to claim 20, wherein the potassium channel activator comprises diazoxide.

22. The method according to claim 17, wherein the potassium channel activator is administered orally.

23. The method according to claim 17, wherein the potassium channel activator is administered at a daily dosage of about 5 mg to about 1200 mg, calculated on a diazoxide active weight basis.

24. The method according to claim 23, wherein the potassium channel activator is administered once, two, or three times a day.

25. The method according to claim 17, wherein the mammal is a human.

26. The method according to claim 25, wherein the mammal is male.

27. The method according to claim 26, wherein the mammal is a hyperinsulinemic obese man.

28. The method according to claim 17, in which the peak plasma insulin level is reduced to about 50 mU/l or less.

29. The method according to claim 17, wherein the potassium channel activator is administered at least once daily for at least one month.

30. A method of treating or preventing obesity in a mammal suffering from or at risk of becoming obese, said method comprising administering to said mammal a therapeutically or prophylactically effective amount of a potassium channel activator.

31. A method of treating or preventing obesity, obesity related disorders and conditions and other disorders and conditions related to weight gain in a mammal in need thereof, said method comprising administering to said mammal a pharmaceutically effective amount of a potassium channel activator.

32. A method of reducing blood pressure, insulin resistance, plasma lipid concentration, or all three in a mammal suffering therefrom, said method comprising administering to said mammal a therapeutically effective amount of a potassium channel activator.
